(19) United States
(12) Patent Application Publication
(10) Pub. No.: US 2005/0085441 A1
(43) Pub. Date: Apr. 21, 2005

(54) Use of soluble monovalent oligosaccharides as inhibitors of HIV-1 fusion and replication

(76) Inventors: James L. Leach, Columbus, OH (US); Stacey Ann Borowicz, Powell, OH (US); Pedro A. Prieto, W. Worthington, OH (US); Balbino Alarcon, Madrid (ES); Ricardo Rueda, Granada (ES); Enrique Vazquez, Granada (ES)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/797,730

(22) Filed: Mar. 10, 2004

Related U.S. Application Data
(63) Continuation-in-part of application No. 10/277,259, filed on Oct. 22, 2002.

(51) Int. Cl. A61K 31/715

(52) U.S. Cl. 514/54; 514/61

(57) ABSTRACT

The subject invention relates to monovalent oligosaccharides and their use, for example, in the treatment and prevention of mammalian disease caused by HIV-1 virus infections. In particular, the oligosaccharides globotriose and lactose may be used alone or in combination to competitively inhibit the formation of the viral fusion complex that occurs upon infection.

### RELATED APPLICATION

This application is a continuation-in-part of pending U.S. application Ser. No. 10/277,259 filed Oct. 22, 2002, and hereby incorporated in its entirety by reference.

### TECHNICAL FIELD

The invention relates generally to compositions containing monovalent oligosaccharides, specifically lactose and globotriose, and to uses thereof. In particular, such compositions have the ability to inhibit HIV-1 infectivity and syncytia formation. Specifically, globotriose and lactose can be used to competitively inhibit formation of the viral fusion complex.

### BACKGROUND OF THE INVENTION

Human Imunodeficiency Virus (HIV) is a retrovirus that causes Acquired Immune Deficiency Syndrome, AIDS. HIV primarily infects cells with CD4 cell-surface receptor molecules, using them to gain entry. It is well established that many of the molecules involved in HIV cell entrance are the keys to future HIV disease treatment and prevention.

Virtually all AIDS cases in United States and Europe are associated with HIV-1 infection. According to the use of cellular coreceptors, there are two different types of HIV-1 strains: the T-tropic—strains that infect preferentially T cells and form syncytia—and the M-tropic—strains that infect preferentially macrophages and do not form syncytia. T-tropic strains use preferentially the chemokine receptor CXCR4 (in addition to CD4) for entering cells. M-tropic strains preferentially use the chemokine receptor CCR5 (in addition to CD4) for entering cells. HIV-2, which was identified years later from West African AIDS patients, is only partially homologous to HIV-1 and genetically more closely related to the Simian Immunodeficiency Virus (SIV) than HIV-1. The transmission of HIV-1 and HIV-2 are similar; however, HIV-2 transmits less efficiently, particularly via heterosexual and perinatal modes. Furthermore, the mortality rate from HIV-2 infection is only two-thirds that for HIV-1.

The complexity of HIV-1 is evidenced by the vast number of proteins encoded by the virus that give rise to a wide variety of pathogenic mechanisms to sustain infection and resist natural and pharmaceutical defenses. This complexity emphasizes the importance of intervening early in viral transmission and cell entry in order to prevent infection and/or the development of AIDS.

HIV-1 enters permissive cells (CD4⁺) by binding to CD4 receptors on the target cell's surface. The fusion of HIV-1 requires the formation of a tri-molecular complex between the viral protein gp120, CD4 and the appropriate chemokine receptor, either CXCR4 or CCR5 (E. A. Berger, et al., Ann. Rev. Immunol., Vol. 17, pages 657-700, 1999). Recent research has demonstrated that gp120 also specifically interacts with some glycosphingolipids (D. Hammache, et al., J. Biol. Chem.Vol. 273, pages 7967-7971, 1998).

Glycosphingolipids are ubiquitous membrane components of the plasma membrane. All share a common hydrophobic transmembrane skeleton that consists of a fatty acid chain and a sphingosine base. This common structure is bound to a variable hydrophilic oligosaccharide residue that protrudes out to the extracellular space. Glycosphingolipids are classified into three main series, ganglioseries, globoseries and lactoseries, according to their carbohydrate structure (S-I. Hakomori and Y. Igarashi, J. Biochem., Vol. 118 pages 1091-1103, 1995).

Cumulative evidence indicates that, in addition to protein coreceptors, HIV-1 uses cell surface glycosphingolipids, specifically Gb3 and GM3, as co-factors for fusion (D. Hammache et al., J. Virol., Vol.73, pages 5244-5248, 1999). The neutral glycosphingolipid Gb3, or globotriaosylceramide, has a globotriose saccharide head. The acidic glycosphingolipid GM3 has a 3′sialyllactose head. Recent findings have shown that 1) depletion of complex glycoshpingolipids from the membrane of normally fusion-competent target cells renders them impervious to HIV-1 fusion, and further that 2) the addition of Gb3 specifically can rescue the fusion event in these depleted target cells, indicating that Gb3 is required for efficient viral fusion (A. Puri et al., Proc. Natl. Acad. Sci., Vol. 95, pages 14435-14440, 1998; A. Puri et al., Biochem. Biophys. Res. Comm., Vol. 242 pages 219-225, 1998). Other research has demonstrated the involvement of GM3 in the fusion event, although the ability of GM3 in restoring the fusion activity of HIV-1 appears to be lower than that reported for Gb3 (P. Hug et al., J. Virol., Vol. 74 pages 6377-6385, 2000).

Oligosaccharides conjugated to a variety of chemical backbones in such a way as to produce a polyvalent oligosaccharide presentation (multiple globotriose moieties at the end of several arms) have been used to treat diseases caused by Shiga and Cholera toxins. The recognition site for these toxins is the oligosaccharide portion of the globoseries-glycolipid globotriaosylceramide, Gb3, known as globotriose (E. A. Merrit and W. G. J. Hol, Curr. Opin. Struct. Biol., Vol. 5 pages 165-171, 1995). Synthetic oligosaccharide analogues, in the form of multivalent clusters, covalently bound to insoluble silica particles, competitively adsorb the toxins and protect susceptible cells, confirming the potential of carbohydrates as new and viable anti-adhesive therapeutic tools (P. I. Kilov et al., Nature, Vol. 403 pages 669-672, 2000). However, present technology relies only upon a multivalent presentation of Gb3, not on monovalent free oligosaccharides.

Infection by HIV starts after initial entry of HIV into the cells. HIV is primarily spread as a sexually transmissible disease. HIV can be transmitted also by parenteral exposure, which is the most efficient method of transmission, close to 90%. HIV infection can also be acquired as a congenital infection perinatally or in infancy. Neonates acquire HIV infections mainly through vertical transmission; mothers with HIV infection can pass the virus transplacentally, at the time of delivery through the birth canal or through breast milk.

The progression of HIV infection into clinical stages is marked by the appearance of typical opportunistic infections or neoplasms, and by the appearance of syncytia-forming variants of HIV. These syncytia-forming variants, derived from non-syncytia-forming variants have greater CD4 cell tropism and are associated with more rapid CD4⁺ decline. The syncytia-forming variants arise prior to the onset of clinical AIDS, however, appearance of syncytia-forming variants of HIV is a marker for progression of AIDS.

Attempts to cure AIDS have not been met with a high degree of success but have been successful in the management of this viral infection. The strategies to manage AIDS include inhibition of reverse transcriptase with AZT and several strategies directed to the inhibition of the fusion/entry mediated through the use of blocking agents for the CD4 and chemokine receptors, or through the genetic depletion of said receptors (E. A. Berger et al., Ann. Rev. Immunol., Vol. 17, pages 657-700, 1999). Compositions containing modified proteins capable of binding to CD4 receptors have been also described as potential tools to competitively prevent binding of HIV-1 and therefore HIV-1 infection (E. A. Berger et al., Ann. Rev. Immunol., Vol. 17 pages 657-700, 1999; U.S. Pat. No. 5,985,275). However, most of these drugs are very expensive and undesired effects on the host have not been fully assessed, especially in vertical HIV transmission cases. Prophylaxis with anti-HIV agents and caesarean section before labor have reduced only slightly the risk of vertical HIV transmission (Grosch-Worner et al., AIDS Vol. 14 pages 2903-2911, 2000). In addition, the mutation rate and emergence of resistant human immunodeficiency viruses is very significant in regular drug therapies.

In view of the above, there is an urgent need to expand the diversity of compounds with potential biological activity directed to preventing HIV-1 and other syncytia-forming viruses from infecting host cells. Polyvalent oligosaccharides that interfere with the binding of glycosphingolipids or their synthesis can be useful in the prevention or treatment of HIV infection (WO 00/29556) through the same mechanism as they have been effective in preventing several pathogen driven interactions with cell oligosaccharides. However, monovalent oligosaccharides have been never considered as potential tools for preventing or treating HIV-1 infections. The monovalent oligosaccharides of the present invention have been shown to prevent these interactions. The monovalent oligosaccharides in fact prevented fusion of HIV-1 virus with target human cells, thereby preventing replication of the virus in question. Furthermore, these oligosaccharides can be produced in large quantities at a reasonable cost (U.S. Pat. No. 5,945,314) rather than producing complex multivalent carbohydrate compositions.

All U.S. patents and publications referred to herein are hereby incorporated in their entirety by reference.

### SUMMARY OF THE INVENTION

The present invention relates to a method of using monovalent soluble oligosaccharide decoys, specifically globotriose (the saccharide portion of Gb3) and lactose (the saccharide portion of GM3), to competitively inhibit HIV-1 fusion with cell membranes.

Furthermore, the present invention relates to a method of using monovalent soluble oligosaccharide decoys to prevent the formation of syncytia resulting after infection with HIV-1.

Moreover, the present invention relates to a method of preventing infections caused by HIV-1 by administering the monovalent soluble oligosaccharides alone or in combination. The present invention relates also to a method of treating established retroviral infections by administering soluble oligosaccharides alone or in combination.

Also, the present invention relates to a composition comprising at least one monovalent oligosaccharide or, a combination of at least two monovalent oligosaccharides. The monovalent saccharide may be, for example, globotriose or lactose.

The composition of the present invention possesses a number of advantages over prior anti-HIV-preparations. First, the monovalent oligosaccharides can be combined with a resulting synergistic effect that requires the use of lower concentrations of each of the oligosaccharides than when used alone. Second, globotriose, lactose and other relevant oligosaccharide decoys can be synthesized in large quantities for a practical cost. Third, the composition can be administered at high doses intravenously, orally or dermally (as a lubricant or spermicidal to be used pre- or post-coitus) with little to no toxic effects. Fourth, globotriose and lactose can specifically inhibit an early event required for infection by the HIV-1 virus. As such, the present invention represents a means to prevent infection rather than a post-infection tool. The invention is unique among other therapeutic approaches to HIV-1 with respect to this property among others. Fifth, globotriose and lactose may be conjugated to other chemical moieties to enhance oral absorption and therapeutic half-life.

Additionally, the composition of the present invention can be used in several ways, including (a) as a pharmaceutical agent administered intravenously, orally or dermally, (b) conjugated to an acceptable chemical moiety to enhance oral absorption, (c) as an ingredient in foods or food supplements to be consumed orally via feeding or parenterally.

### BRIEF DESCRIPTION OF THE FIGURES

FIG. 1 represents the effect of selected oligosaccharides on HIV-1 T-tropic strain replication in MT-2 cells as evidenced by syncytia formation and Ag p24 release (insert). B=globotriose; C=lactose.

FIG. 2 shows the effect of selected oligosaccharides on HIV-1 replication in MT-2 cell line at days 3 through 12 post-infection. A=lacto-N-tetraose; B=globotriose; C=lactose.

FIG. 3 represents the synergistic effect of globotriose (B) and lactose (C) on HIV-1 replication in MT-2 cell line. Numbers after the initials represent concentration in mM.

FIG. 4 graphically shows the effect of selected oligosaccharides on the replication of clinical isolate 1936 in MT-2 cells. A=lacto-N-tetraose; B=globotriose; C=lactose. Numbers after the initials represent concentration in mM.

FIG. 5 shows the effect of selected oligosaccharides on the replication of clinical isolate ME46 in MT-2 cells. B=globotriose; C=lactose. Numbers after the initials represent concentration in mM.

FIG. 6 represents the effect of selected oligosaccharides on the replication of clinical isolate CBL23 (HIV-2) in MT-2 cells. B=globotriose; C=lactose. Numbers after the initials represent concentration in mM.

FIG. 7 shows the effect of selected oligosaccharides on CD4 expression in MT-2 cells, 3 and 5 days post-infection with isolate NL4.3. A=lacto-N-tetraose; B=globotriose; C=lactose. Numbers after the initials represent concentration in mM.

FIG. 8 shows the effect of selected oligosaccharides on the replication of HIV-1 M-tropic strain Ba-L in MT-2 cells. A=lacto-N-tetraose; B=globotriose; C=lactose. Numbers after the initials represent concentration in mM.

FIG. 9 graphically presents the effect of selected oligosaccharides on the entrance of HIV-1 isolate pNL4.3luc in MT-2 cells. Upper panel: pNL4.3luc virus pseudotyped with T-tropic envelope of HXB2 (HIV). Lower panel: pNL4.3luc virus pseudotyped with protein G of vesicular stomatitis virus (VSV). B=globotriose; C=lactose. Numbers after the initials represent concentration in mM.

FIG. 10 graphically shows the inhibition of HIV replication in SCID-hu-PBL mice in the presence of selected oligosaccharides. B=globotriose; C=lactose; ST=untreated.

FIG. 11 graphically shows confirmatory results on the inhibition of HIV replication in SCID-hu-PBL mice in the presence of globotriose in vivo.

### DETAILED DESCRIPTION OF THE INVENTION

As noted above, the subject matter of the present invention relates to compositions comprising at least one monovalent oligosaccharide and a pharmaceutically acceptable carrier, which can be used to prevent or treat infections caused by HIV-1 virus.

a) Definitions and related information

As used herein, the term “monovalent” means a single chemical unit with a free anomeric carbon which is not conjugated or bound to an inert matrix and which lacks a synthetic linking arm. An “oligosaccharide” is a sugar molecule that contains approximately 2-10 sugar units. The sugar units (i.e., (CH₂O)n) in an oligosaccharide are connected by glycosidic linkages. Examples of the monovalent oligosaccharides of the present invention which may be used in the treatment and prevention of, for example, HIV-1 infection include, for example, the trisaccharide globotriose (also known as galactose α1-4galactose β1-4glucose) and the disaccharide lactose (also known as o-β-D-galactopyranosyl-(1-4)-β-D-glucopyranose). It should be noted, however, that the use of any monovalent oligosaccharide that has the ability to competitively inhibit binding of viral gp120 to the cellular target(s) is considered to fall within the scope of the present invention. Such oligosaccharides are all readily soluble.

As used herein, a “retrovirus” is a virus that has RNA as its genome but needs to transcribe it to DNA during its replicative cycle within the infected cell. When a retrovirus infects a cell, it must use its reverse transcriptase enzyme to transcribe its RNA to host cell proviral DNA. This DNA becomes integrated in the host chromosomes, and it is this proviral DNA that directs the cell to produce additional virions that are released subsequently. Retroviruses in general contain three major genes: Gag, Pol, and Env. The major structural components coded by Env include the outer envelope glycoprotein gp120 and the transmembrane glycoprotein gp41 derived from glycoprotein precursor gp160. In the case of HIV-1, the virus enters cells by binding to the cellular CD4 receptor, followed by gp120-gp41-mediated fusion of the viral and target cell membranes. “MT2 cell” is a human T lymphotropic virus—transformed cell expressing the CD4 receptor.

As used herein, “CD4⁺ cells” means cells expressing the CD4 receptor on the cell surface. CD4 is the primary receptor for the binding of the viral outer envelope glycoprotein, Env. After binding, fusion of viral envelope and CD4⁺ cells occurs, leading to entry of the viral particle into the cell.

“Chemokine receptors” as used herein, means co-receptors required for HIV-1 entry. This notion resulted from the fact that CD4 expression was not sufficient to explain HIV-1 entry in target cells. The chemokine receptors most frequently used as HIV-1 receptors are CXCR4 and CCR5 for T-tropic and M-tropic strains of HIV-1, respectively.

A “syncytia-forming virus” is a virus that, after infecting susceptible cell cultures, produces cytopathogenic effects in the form of syncytia. HIV-1, for example, is a syncytia-forming virus.

“Syncytia” or “giant cells” are large masses containing up to 100 nuclei and are believed to result from the fusion of virus-infected cells with non-infected cells. The formation of syncytia may be analyzed, for example, by phase contrast microscopy using specific staining, for the visualization of nuclei.

Another indicator of the progression of HIV infection, in addition to the formation of syncytia, is the presence of p24 antigen. Although only about 60% of HIV-infected persons develop p24 antigenemia prior to the onset of clinical AIDS, the p24 antigen is a highly specific predictor of the progression of HIV infection both in vitro and in infected patients. Viral replication in cells is followed by the measurement of HIV p24 antigen using an antigen capture immunoassay. A decreased signal is an indication of a retarded or decreased release of p24. Said decrease in the presence of oligosaccharides is an indication of the inhibitory effect of the oligosaccharides on viral replication.

As used herein, the term “T-tropic” refers to HIV-1 isolates that show efficient infectivity for continuous CD⁴⁺ T cell lines, but poor infectivity for macrophages. This phenotype was originally observed with isolates that had been produced in the laboratory (X4 and pNL strains) and are generally syncytia-forming strains. T-tropic HIV-1 isolates infect cells that express the co-receptor CXCR4. A “clinical T-tropic isolate” such as isolate 1936 (Muñoz-Fernández et al, Pediatr. Res., Vol. 45, pages 597-602, 1996) and ME46 (NIH-AIDS Research and Reference Reagent Program), is a T-tropic virus obtained from infected donors and maintained in primary cells such as human T lymphoblasts.

The term “M-tropic” refers to HIV-1 isolates (such as the Ba-L strain, (Gartner et al, Science Vol. 233, pages 215-219, 1986)) that are non-syncytia-forming and that infect primarily macrophages.

Cells are infected at different “MOI”, which means multiplicity of infection. In particular, the MOI is the number of virus particles or infectious units adsorbed per cell.

In terms of mechanism of action, the antiviral activity of the monovalent oligosaccharides of the present invention is not mediated by a down regulation or masking of the CD4 or chemokine receptors. The monovalent oligosaccharides of the present invention are thought to act as decoys and prevent the binding of the viral protein gp120 to the cellular target, thereby preventing HIV-1 fusion with the target cell. Specifically, the unconjugated monovalent oligosaccharides of the present invention may competitively inhibit the viral fusion by serving as alternative receptors for the viral gp120, as opposed to the oligosaccharide heads of the glycosphingolipid surface on the target cells.

In view of the above, the monovalent oligosaccharides of the present invention present several advantages over other existing approaches. First, the antiviral activity of the unconjugated monovalent oligosaccharides is effective not only as an important treatment tool, but also as a preventive measure by blocking the initial binding of the viral protein to the cellular target. By administering the oligosaccharides, the first contact of the viruses with susceptible cells will not occur, preventing the entrance and further replication of virions. Further, it should be noted that the monovalent oligosaccharides described herein may be used to inhibit not only HIV-1 interactions but also other pathogen-driven interactions, currently inhibited by complex polyvalent carbohydrate compounds, as well.

Second, another advantage of the oligosaccharides of the present invention is the synergy of effects, resulting from the combination of globotriose and lactose. When administered together, the inhibitory effect on HIV-1 replication is increased to more than double compared to the effect of each oligosaccharide alone.

Third, with respect to production, the monovalent oligosaccharides of the present invention may be made either recombinantly or synthetically, using techniques known in the art, rather than producing complex polyvalent arrays of oligosaccharides. For example, U.S. Pat. No. 5,945,314, incorporated in its entirety herein by reference, describes a method of synthetically producing oligosaccharides.

Further, in order to treat or prevent HIV-1 infection, including vertical transmission at the time of delivery, the monovalent oligosaccharides of the present invention can be administered to adults, infants or newborns, enterically or parenterally. For example, the oligosaccharide may be utilized in a rehydration or hydration solution provided either orally (e.g., Pedialyte® or Equalyte®, Abbott Laboratories, IL) or intravenously (e.g., saline/D5W).

Additionally, one or more oligosaccharides of the present invention can be utilized in pharmaceutical compositions also comprising a pharmaceutically acceptable carrier. A “pharmaceutically acceptable carrier” is any compatible, non-toxic substance suitable for delivering the oligosaccharide(s) to the patient. Examples include sterile water, alcohol, fats, waxes, inert solids, phosphate buffered saline, oils, or water/oil emulsions. The composition may be in either in the form of a tablet, a capsule, an intravenous liquid or injectable, or a dermal cream. The dosage of the composition as well as the form and method of administration may be readily determined by one of ordinary skill in the art in view of such factors such as age, weight, immune state, etc.

Further, the oligosaccharides may be administered as part of an antibiotic or antiviral “cocktail” comprising several antibiotic agents and/or antiviral agents (e.g. Norvir®, Abbott Laboratories, IL), or in conjunction with other agents being used to treat or prevent the symptoms caused by HIV-1 infection. This is especially important in groups most at risk of infection, through promiscuous sexual activity, drug use and perinatal infection.

b) General Methods

In general, and as will be elucidated in detail in the following examples, the mechanism of action of the monovalent oligosaccharides on HIV-1 replication was studied on MT-2 cells infected with HIV-1. Increasing concentrations of oligosaccharides were added to the culture media and their inhibiting effects were compared to controls without oligosaccharides. Viral replication was monitored by measuring viral p24 antigen in the supernatant of the cell culture using an antigen capture immunoassay. Similarly, formation of syncytia was analyzed by phase contrast and fluorescence microscopy with staining to visualize the nucleus.

The inhibitory effects of globotriose and lactose on HIV-1 replication were also tested in monocytes cultures. Similarly to MT-2 cells, viral replication in monocytes was measured by release of viral antigen p24 in controls and in the presence of increasing concentrations of oligosaccharides.

The following examples are offered for illustrative purposes only, and are not intended to limit the scope of the present invention in any way.

### EXAMPLE 1

### Globotriose Prevents Replication of Laboratory Isolate of HIV-1 T-Tropic Strains in MT-2 Cells

As a first approach to study the mechanism of action of globotriose, an experiment involving one-round of replication was performed. The CD4⁺ human T lymphotrophic virus (HTLV-1) transformed cell line MT-2 was obtained from the National Institute for Biological Standards and Control, Medical Research Council, UK (repository reference ARP014). The cells were maintained in RPMI-1640 plus 5% heat-inactivated fetal bovine serum (FBS). MT-2 cells were incubated with HIV-1 at low (0.001 pfu/cell) or high (2.5 pfu/cell) multiplicity of infection (MOI) in the presence of different concentrations of drugs for different periods of time. High-titer stocks of HIV-1 laboratory strain NL4.3 (kindly provided by Dr. J. Alcamí, Hospital Doce de Octubre, Madrid, Spain) were prepared (Adachi et al., J. Virol. Vol. 59, pages 284-291, 1986), and the titers were determined using the endpoint dilution method of Kärber (Arch. Exp. Pathol. Pharmakol., Vol. 162, pages 480-483, 1931).

Briefly, 10⁶ MT-2 cells were infected with the HIV-1 strain at a MOI of 0.001 in a final volume of 5 ml of complete medium (RPMI-1640 plus 5% heat-inactivated FBS). After 3-4 days, 75% of the cells had formed syncytia (i.e., cells that had fused into giant multinucleated cells). 30×10⁶ MT-2 cells in 30 ml of complete medium were added to the culture and incubated again until 75% of the cells had formed syncytia in the culture. The cells were then centrifuged at 1200 rpm for 10 minutes; the supernatant was filtered through a 0.4 μm pore filter and titrated. The formation of syncytia was analyzed by phase contrast and fluorescence microscopy with 1 μg/ml Hoechst 33258 (Sigma Chemical, Co. St. Louis, Mo.) for 7 minutes on ice after staining to visualize the nuclei.

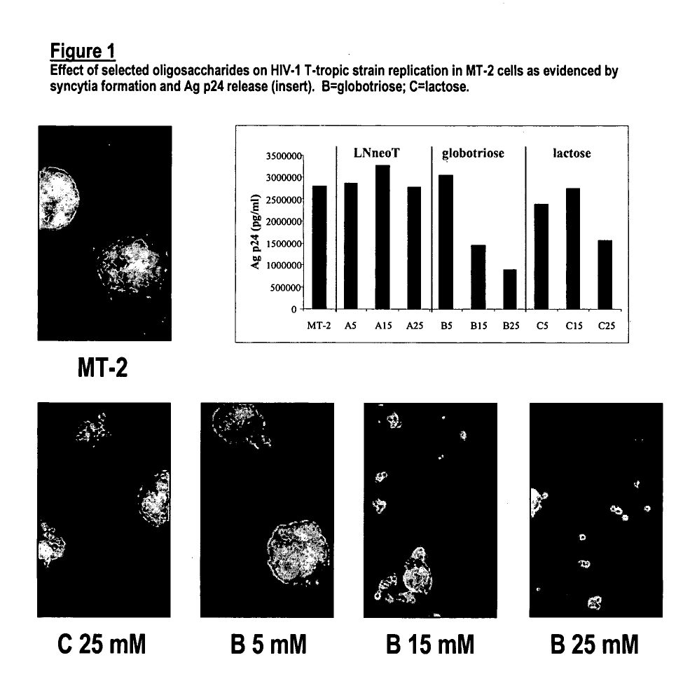

Three different concentrations (0.5-50.0 mM) of the three oligosaccharide compounds globotriose, lactose and lacto-N-tetraose were used. Globotriose completely inhibited the formation of syncytia, both in terms of number and size of syncytia, at a concentration of 25 mM. At lower concentrations, globotriose still had an effect on the formation of syncytia since the size of these syncytia (i.e., the number of nuclei as an indication of the number of fused cells) was decreased. Lactose had an effect although weaker than that exhibited by globotriose (FIG. 1). Since syncytia are formed after fusion of HIV-1-infected cells that express the envelope protein on the cell surface with non-infected CD4⁺ cells, the results, as shown in FIG. 1, indicate that oligosaccharides directly prevent HIV fusion with target cell membranes, therefore preventing the formation of syncytia.

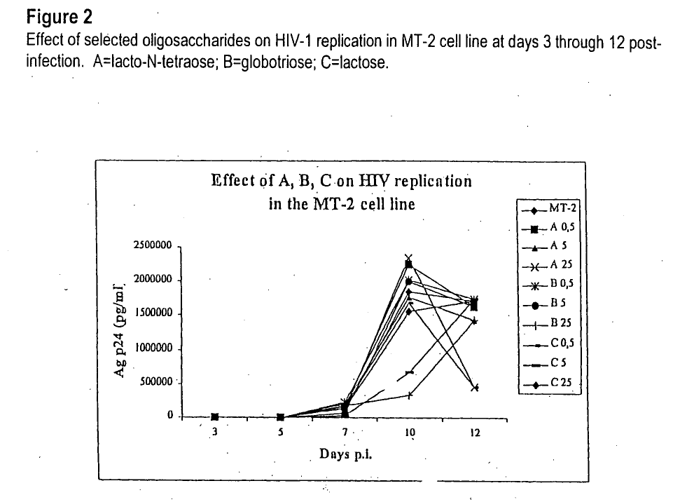

Additionally, viral replication was monitored each 3-4 days by measuring release of viral p24 antigen in the supernatant of the cultures using an antigen capture immunoassay. The release of antigen p24 was inhibited by globotriose at a concentration of 25 mM but not at 5 mM or lower. The inhibitory effect was maximum at days 3 and 7, but it was recovered at day 10 post-infection. Lactose had an effect although less potent than globotriose. Lacto-N-tetraose was ineffective (FIG. 2).

Toxicity of high concentrations of oligosaccharides was assessed by counting MT-2 cells (100,000 cells/ml) after incubation with globotriose and lactose at concentrations ranging from 0.5-50 mM. After 2, 4 and 7 days, the number of cells was counted in a Neubauer chamber (Brand-Blau Brand, Germany) and their viability was estimated by Tripan Blue exclusion. At a concentration of 50 mM, globotriose was the only treatment to cause cell toxicity. Globotriose at 50 mM inhibited the proliferation of MT-2 cells and decreased their viability to 60% after 4 days of culture (data not shown). Lactose at concentrations as high as 50 mM did not cause significant toxicity. These results indicate that globotriose may be toxic at this high concentration. Lower concentrations of globotriose (including 25 mM) showed no toxic effect on MT-2 cells.

### EXAMPLE 2

### Synergistic Effects of Lactose and Globotriose

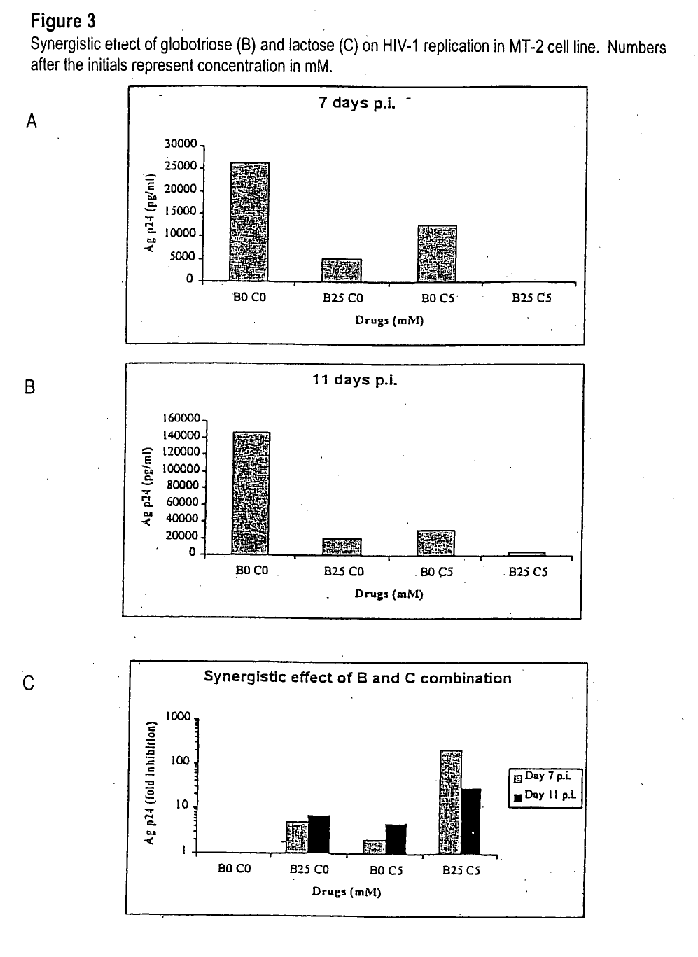

Based on the anti-HIV-1 activity of both globotriose and lactose, the effect of their combination was assessed by following the procedures indicated in Example 1. The combination of 25 mM globotriose and 5 mM lactose exerted a synergistic effect. At day 7 post-infection, 25 mM globotriose reduced by 5 fold the production of p24, and 5 mM lactose reduced it by 2 fold. The combination of both oligosaccharides at the same concentrations completely inhibited viral replication reducing p24 release by 222 fold (FIG. 3). This unexpected result represents an advantage over other therapies already in existence. The possibility of using both oligosaccharides simultaneously results in an enhanced therapeutic activity with no collateral toxic effects as may be present using other therapeutic approaches.

### EXAMPLE 3

### Globotriose Prevents Replication of Clinical Isolates of HIV-1 T-Tropic Strains in MT-2 Cells

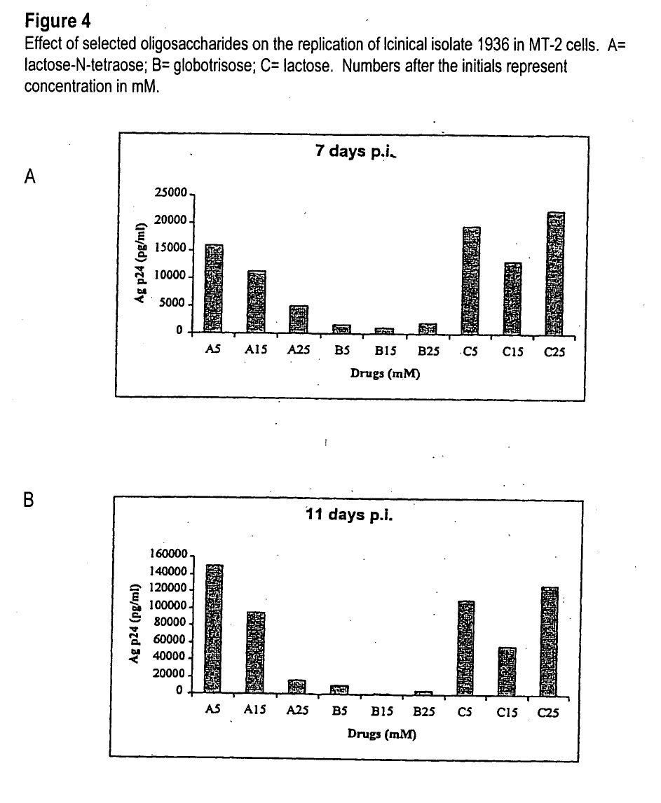

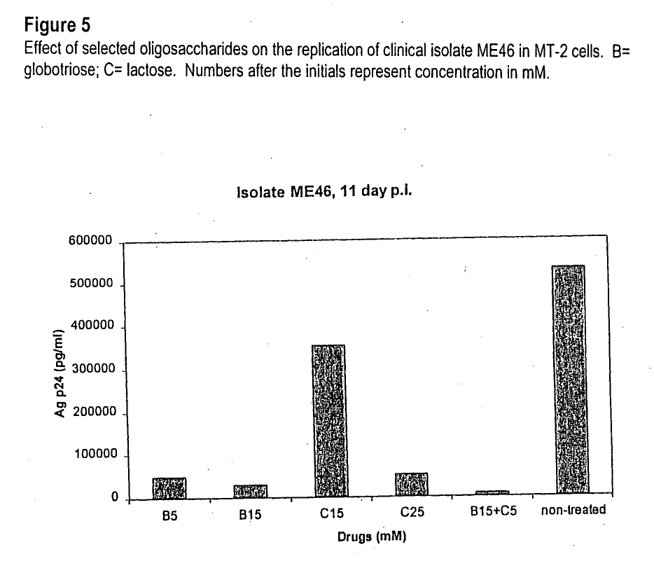

The effects of the globotriose and lactose were also tested on the replication of two T-tropic clinical isolates of HIV-1, i.e., 1936 and ME46. MT-2 cells were infected as described above, and the course of infection was followed by the release of p24. Globotriose inhibited more than ten-fold the replication of isolates 1936 (FIG. 4) and ME46 (FIG. 5) at concentrations of 5 mM and above. Contrary to globotriose, lactose practically did not affect replication of isolate 1936 at a concentration of 25 mM (FIG. 4). Nevertheless, at this concentration, lactose inhibited by ten-fold the replication of isolate ME46 but only had a minor effect at 15 mM (FIG. 5). A combination of 15 mM globotriose and 5 mM lactose, that was previously shown to have synergistic effects against the replication of HIV-1 strain NL4.3 (see Example 2), was considerably more potent than globotriose or lactose alone against strain ME46.

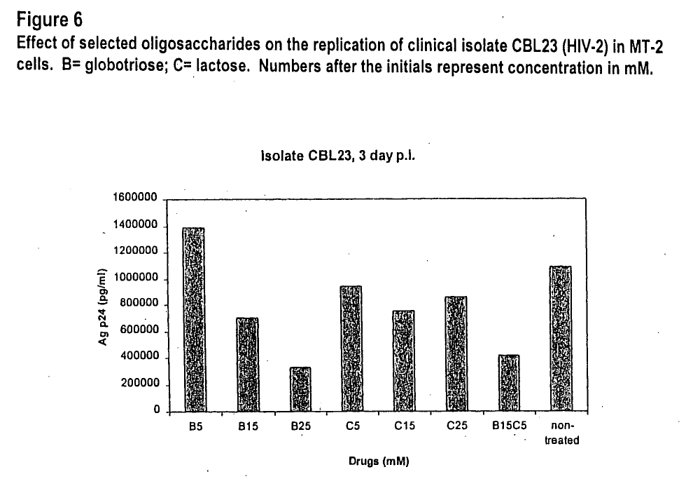

The effect of the oligosaccharides was also tested on the replication of CLB23, a T -tropic clinical isolate of HIV-2. Globotriose inhibited replication of HIV-2 isolate CBL23 although the effect was much weaker than for the HIV-1 isolates (FIG. 6). Lactose was basically non-effective, and the combination of 15 mM globotriose and 5 mM lactose potentiated the inhibitory effect but, even so, the inhibition was modest (FIG. 6). These results indicate that globotriose, and particularly the globotriose plus lactose combination, inhibited the replication of two clinical isolates of HIV-1 but the antiviral effect against the HIV-2 isolate was more modest.

### EXAMPLE 4

### Effect of Oligosaccharides on CD4 Expression

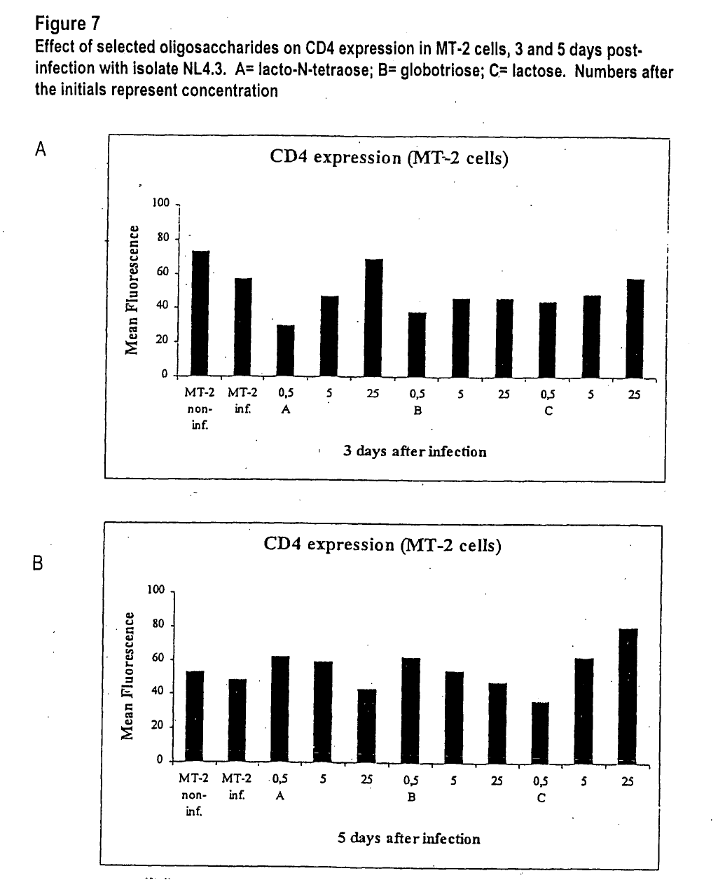

Some glycosphingolipids inhibit HIV-1 replication by inducing the down-regulation of CD4 receptors on HIV-1 sensitive cells. To determine whether CD4 expression was altered in the presence of the oligosaccharides, HIV-1 infected cells treated with the oligosaccharides were washed in PBS (Phosphate Buffered Saline) and incubated on ice with FITC-conjugated anti CD4 antibody Leu3a (BD Biosciences, Heidelberg, Germany) for 30 minutes. The samples were washed, fixed with 1% paraformaldehyde and analyzed by flow cytometry. As shown in FIG. 7, expression of CD4 receptors remained at similar levels independent of the dose and of the compound used in the experiment. These results demonstrate that the antiviral activity of the oligosaccharides is not mediated by a down regulation or masking of the CD4 receptors, but rather by preventing HIV-1 fusion with the target cell.

### EXAMPLE 5

### Effect of Oligosaccharides on Replication of M-Tropic Strain of HIV-1 in Monocytes

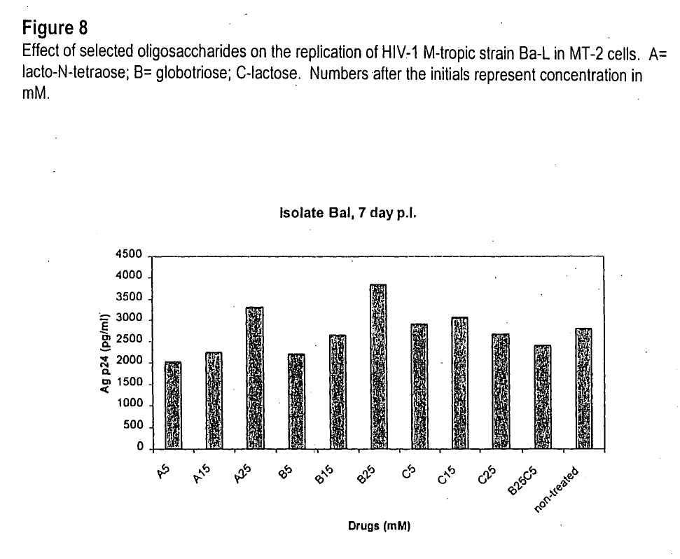

Human peripheral blood monocytes were isolated from whole blood of healthy donors by Ficoll Hypaque density gradient centrifugation (Pharmacia Fine Chemicals, Uppsala, Sweden). The mononuclear cell fraction was incubated in culture dishes for 24 hours at 37° C. Non-adherent cells were discarded, and adherent cells were maintained in RPMI medium plus 5% fetal bovine serum for 6 days. The cells were determined to be more than 80% positive for the monocytic marker CD14 by flow cytometry. Stocks of the M-tropic strain Ba-L (Gartner et al., Science Vol. 233 pages 215-219, 1986) were prepared using human peripheral blood monocytes. Adherent monocytes were subsequently infected at a MOI of 0.001 with Ba-L in the presence of globotriose (B), lactose (C) and lacto-N-tetraose (A) at concentrations of 0.5, 5 and 25 mM. Viral antigen p24 release was measured as an estimate of viral replication. Unlike its effects on the replication of the T-tropic strain of HIV-1, pNL4.3, globotriose did not inhibit the replication of the R5 M-tropic strain Ba-L in cultured human peripheral blood monocytes. Lactose and lacto-N-tetraose were also ineffective. To determine whether the absence of effect was due to the cell type, a MT-2 cell clone transfected with CCR5 co-receptor was infected with strain Ba-L. The result confirmed that globotriose, lactose or the combination of both were not inhibitory of viral replication of M-tropic strains (FIG. 8).

These findings emphasize the potential usefulness of oligosaccharides in treating or preventing infection by syncytia-forming types of viruses.

### EXAMPLE 6

### Globotriose and Lactose Inhibit the Early Steps of HIV-1 Replication in Infected MT-2 Cells

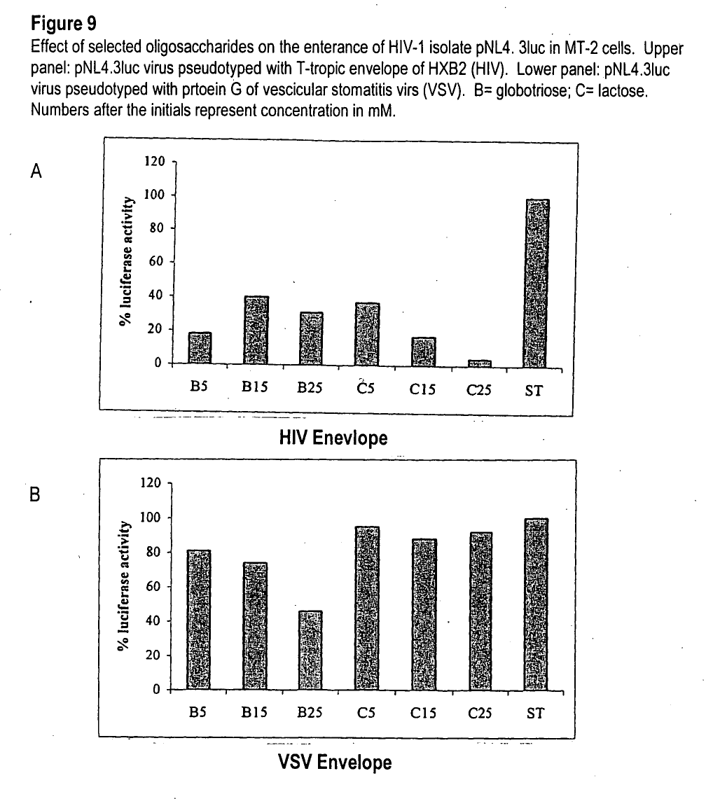

To investigate the mechanism of action, a single-cycle experiment of HIV expression in the presence of different concentrations of globotriose and lactose was performed. For this experiment, human 293T cells were co-transfected with the NL4.3luc construct and either a construct expressing the HIV-1 T-tropic envelope protein of HXB2 (HXB2) or a construct expressing the vesicular stomatis virus (VSV) G protein. The NL4.3luc construct encodes for the complete HIV-1 genome except for the env gene and instead has a luciferase gene reporter. Cells transfected with this construct together with a variety of construct expressing viral envelope proteins can thus produce HIV virions with a variety of coats. Virions produced in this way can enter into cells, integrate and express the luciferase gene in an envelope-dependent manner. When supernatants of 293T cells transfected with the NL4.3luc construct and the HIV HXB2 envelope protein were incubated with MT-2 cells in the presence of globotriose and lactose, expression of the luciferase reporter was inhibited (FIG. 9). However, when HIV virions were coated with the VSV G protein, no effect was observed except for the highest concentration of globotriose.

These results show that globotriose and lactose inhibit integration and expression of the luciferase gene reporter in an envelope-dependent manner. Consequently, these results suggest that globotriose and lactose inhibit the HIV-1 envelope protein-dependent functions and therefore suggest that they inhibit the entry step.

### EXAMPLE 7

### Globotriose and Lactose Inhibit HIV-1 Replication In Vivo in a Mouse SCID-huPBL Model

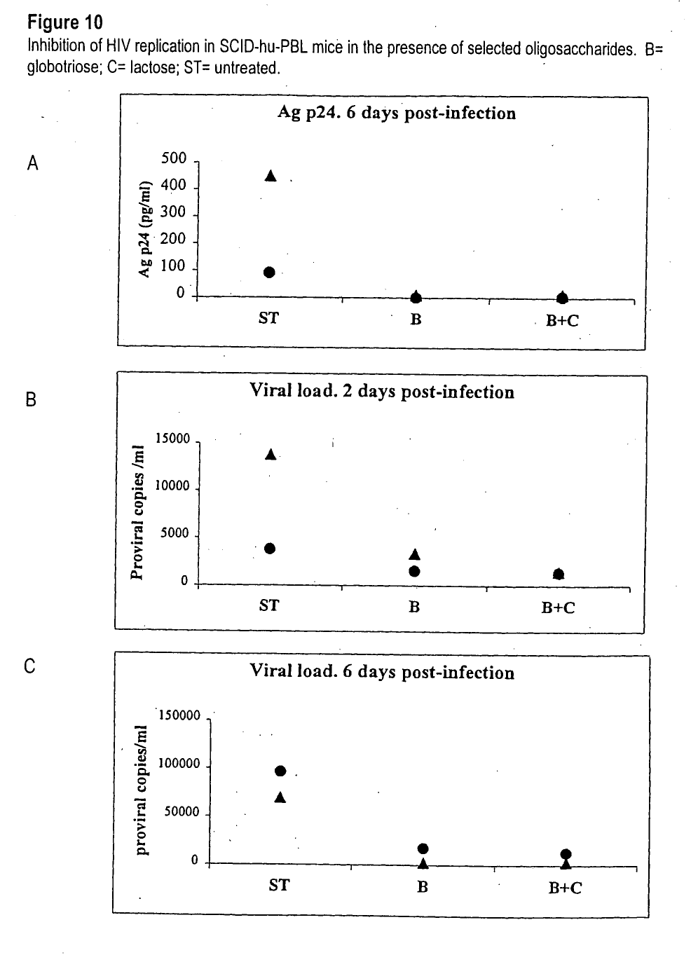

In a first attempt to study the effect of the above sugars in an animal model, NOD-SCID mice (Prochazka et al, Proc. Natl. Acad. Sci. USA Vol. 89 pages 3290-4, 1992; Christianson et al, Diabetes Vol.42 pages 44-45, 1993; Schultz et al, J. Immunol. Vol. 154 pages 180-91, 1995) were implanted with human PBMC in the peritoneum and infected with HIV-1 two weeks later. Twelve mice were reconstituted by intraperitoneal injection of 20×10⁶ freshly isolated normal human PBMCs. Two weeks later, the mice were injected intraperitoneally with 20×10⁶ lymphoblasts from the same donor. After 24 h, 10⁶ lymphoblasts infected with HIV-1 (strain NL4.3) at a multiplicity of infection of 1 were injected i.p. Globotriose (15 mg/mouse) was administered i.p. in a single dose to a group of 4 infected mice (B). Globotriose (15 mg/mouse) and lactose (3.5 mg/mouse) were administered i.p. to another group of 4 mice (B+C), and the rest were left untreated (ST). Mice were sacrificed 2 and 6 days post infection. The course of infection was followed according to the release of p24 viral antigen and viral load detected in the peritoneal wash. The evolution of infection was followed at day 6 by measuring p24 concentration and viral load, and at day 2 by measuring viral load (p24 concentration was too low at this point). Two animals were sacrificed for each experimental point (FIG. 10, each symbol represents an animal, two symbols per group, two mice per group). The results show that globotriose strongly inhibited HIV-1 replication in this animal model at both time points. However, a clear benefit of the globotriose plus lactose combination was not observed in this experiment.

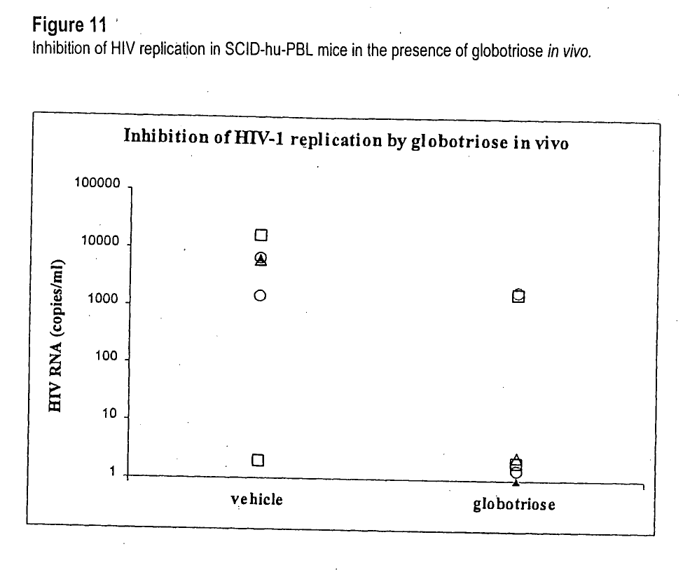

The experiment was repeated to assess the anti-HIV activity of globotriose in the NOD-SCID huPBL model. In this occasion, PBM from three different human donors were implanted in the peritoneum of a total of twelve mice. Globotriose was administered in a single dose at the beginning of the infection, and its effect on p24 and viral load was examined at day 6. Although the levels of p24 and viral load in the control group were lower in this experiment, the treatment with globotriose resulted in diminished replication of HIV-1 (Table I). This was further confirmed in a third experiment using two groups of mice, i.e., treated with globotriose and untreated. (FIG. 11, each symbol represents an animal, six symbols per groups, six mice per group).

In summary, these examples demonstrate that the monovalent oligosaccharides of the present invention have a potent inhibitory effect against replication of laboratory and clinical T-tropic strains HIV-1 at non-toxic concentrations. Globotriose has a more moderate effect, against a clinical isolate of HIV-2.

Additionally, the examples indicate that the monovalent oligosaccharides of the present invention can be used alone or in combination. The antiviral effect was multiplied in the presence of non-effective doses of lactose. The combination of low concentrations of the two oligosaccharides of the present invention results in a greater anti-viral activity than that observed upon using either oligosaccharide alone, without increasing potential collateral effects that could result by increasing the concentration of each one individually However, globotriose alone (or in combination with lactose) was not inhibitory for a M-tropic strain of HIV-1, suggesting that these sugars inhibit CXCR4-dependent but not CCR5-dependent strains. In regard to the mechanism of action, both globotriose and lactose inhibit the entry step.

These results also demonstrate the effectiveness of globotriose in inhibiting the replication of HIV in an animal model.

Further, these results also suggest that the monovalent oligosaccharides of the present invention may have a broad spectrum of activity not only against clinically relevant HIV-1 strains, but also against other syncytia-forming viruses.

In conclusion, the monovalent oligosaccharides of the present invention can be used alone or in combination not only as a therapeutic tool to treat established infections, but may also be used as a preventive treatment in individuals at risk for contracting HIV-1 or other syncytia-forming viruses.

1. A method of inhibiting the fusion of a retrovirus with cell membranes, comprising the step of administering a composition comprising at least one monovalent oligosaccharide to a mammal in an amount sufficient to effect said inhibition of fusion.

2. A method of inhibiting retrovirus-mediated syncytia formation, comprising the step of administering a composition comprising at least one monovalent oligosaccharide to a mammal in an amount sufficient to effect said inhibition of retrovirus-mediated syncytia formation.

3. The method of claim 1 or 2, wherein the retrovirus is a Human Immunodeficiency Virus (HIV).

4. The method of claim 3 wherein the HIV is Human Immunodeficiency Virus type 1 (HIV-1).

5. The method of claim 1 or 2, wherein the retrovirus is a syncytia-forming virus.

6. The method of claim 5 wherein the syncytia-forming virus is a HIV-1 variant.

7. The method of claim 1, wherein said composition comprises at least two of said monovalent oligosaccharides in an amount sufficient to synergistically augment said inhibition of fusion.

8. The method of claim 2, wherein said composition comprises at least two of said monovalent oligosaccharides in an amount sufficient to synergistically augment said inhibition of syncytia formation.

9. A method of preventing an infection in a mammal caused by a retrovirus, comprising the step of administering a composition comprising at least one monovalent oligosaccharide to a mammal, wherein said composition is administered in an amount sufficient to effect said prevention.

10. The method of claim 9 wherein said composition comprises at least two of said monovalent oligosaccharides in an amount sufficient to effect said prevention.

11. A method of treating an infection in a mammal caused by a retrovirus, comprising the step of administering a composition comprising at least one monovalent oligosaccharide to a mammal, wherein said composition is administered in an amount sufficient to effect said treatment.

12. The method of claim 11 wherein said composition comprises at least two of said monovalent oligosaccharides in an amount sufficient to effect said treatment.

13. The method of claim 9 or 11 wherein said retrovirus is a Human Immunodeficiency Virus (HIV).

14. The method of claim 13 wherein said HIV is the Human Immunodeficiency Virus type 1 (HIV-1).

15. A method for preventing transmission of HIV in a mammal, comprising the step of administering a composition comprising at least one monovalent oligosaccharide to said mammal, wherein said composition is administered in an amount sufficient to prevent said transmission.

16. The method of claim 15 wherein said composition comprises at least two of said monovalent oligosaccharides in an amount sufficient to effect said prevention of said transmission.

17. The method of claim 15, wherein the transmission is perinatal vertical transmission.

18. A composition comprising at least one monovalent oligosaccharide, wherein said at least one monovalent oligosaccharide inhibits interaction of CD4 receptors, viral gp120 and membrane glycolipids.

19. The composition of claim 18 wherein said at least one monovalent oligosaccharide is selected from the group consisting of globotriose and lactose.

20. The composition of claim 19 wherein said at least one monovalent oligosaccharide is globotriose.

21. The composition of claim 20 wherein said globotriose is present in a concentration between 5 mM and 25 mM.

22. The composition of claim 19 wherein said at least one monovalent oligosaccharide is lactose.

23. The composition of claim 22 wherein said lactose is present in a concentration between 5 mM and 25 mM.

24. The composition of claim 18 wherein said composition is selected from the group consisting of a pharmaceutical composition and a nutritional composition.

25. The composition of claim 24 wherein said composition can be administered by a route selected from the group consisting of parenteral administration, enteral administration, and dermal administration.

26. The composition of claim 25 wherein said parenteral administration is intravenous.

27. The composition of claim 25 wherein said enteral administration is oral.

28. The composition of claim 25 wherein said dermal administration is local.
